(19) United States
(12) Patent Application Publication
(10) Pub. No.: US 2002/0059657 A1
(43) Pub. Date: May 16, 2002

(54) Homeobox binding sites and their uses

(76) Inventors: Michael Freeling, Berkeley, CA (US); Mark Lubkowitz, Colchester, VA (US); Petra Bauer, Hendersleben (DE)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/823,109

(22) Filed: Mar. 30, 2001

Related U.S. Application Data
(60) Provisional application No. 60/193,179, filed on Mar. 30, 2000.

(51) Int. Cl. C12N 15/82; A01H 5/00

(52) U.S. Cl. 800/278; 800/290; 800/320.1

(57) ABSTRACT

This invention provides recombinant expression cassettes comprising a homeobox binding site having a sequence at least substantially identical to ATTATTACATGNG or ATTATTATTACATGNG operably linked to a heterologous plant target polynucleotide sequence. The recombinant expression cassette may be incorporated in a recombinant plasmid or integrated into the genome of a transgenic plant.

### CROSS REFERENCE TO RELATED APPLICATIONS

This application claims priority to U.S. Ser. No. 60/193,179, filed Mar. 30, 2000, which is incorporated herein by reference.

### STATEMENT AS TO RIGHTS TO INVENTIONS MADE UNDER FEDERALLY SPONSORED RESEARCH AND DEVELOPMENT

This invention was made with Government support under Grant Nos. RO1-GM42610 and F32 GM19394 awarded by the National Institutes for Health. The Government has certain rights in this invention.

### FIELD OF THE INVENTION

This invention relates to plant genetic engineering. In particular, it relates to the identification of homeobox binding sites and their use to modulate gene expression in plants.

### BACKGROUND OF THE INVENTION

The ability to regulate gene expression in different plant tissues is useful for a variety of applications. For example, manipulation of the relative number or size of different organs or tissues in plants is particularly useful. Ectopic expression of specific genes can result in an increase or decrease in the number and/or size of a specific organ. Such manipulations have important agricultural advantages.

Homeobox genes are one major class of genes that encode proteins that affect tissue-specific gene expression (Affolter, M., Schier, A. and Gehring, W. J. (1990) Curr. Opin. Cell. Biol. 2:485). Homeobox genes constitute a family of genes that play essential roles during development. These genes are involved, in particular, in conferring specific identities to different regions or cells.

Homeobox genes were first identified in the fly, Drosophila melanogaster, on the basis of the dramatic phenotypes that their mutations produced. Homologues have also been isolated from other animal species and more recently from plants. Homeobox genes are characterized by the presence within each gene of a well conserved sequence, the homeobox, that encodes a DNA binding domain called the homeodomain. The homeodomain-containing proteins encoded by the homeobox genes are thus capable of binding to specific DNA sequences and perform their role in development by acting as transcription factors. The downstream genes directly regulated by homeodomain-containing proteins are however still largely unidentified (Mannervick, M. (1999) Bioessays. 4:267).

Homeobox genes have been grouped in several families, based on different characteristics, including variations in the homeodomain primary sequences and structures that result in different preferred DNA binding sites.

One family of homeobox genes is the TALE superclass. This family includes genes from the MEIS, the PBC, the Iroquois, the TGIF and the plant KNOX class of genes (Burglin, T. R. (1997) Nucleic Acids Res. 25:4173). Genes belonging to this family have been identified in worms, Drosophila, Xenopus, Zebra-fish, chicken, mice, humans, fungi and plants. In animals, the TALE genes are involved in various developmental processes, including neural and heart development, and have also been implicated in neoplasias and in particular leukemia. In plants, KNOX genes have been identified in numerous plants, both monocots, such as, rice and maize, and dicots, such as tomato, they seem to be primarily involved in shoot and leaf development.

In both monocot and dicot plants, leaf initiation requires the early specification of founder cells in the meristem (Poethig, S. in Contemporary Problems in Plant Anatomy, R. A. White and W. C. Dickinson, Eds. (Academic Press, New York, 1984),pp. 235-259). The acquisition of founder cell identity appears to be determined by the KNOX class of homeobox genes that are normally expressed in the meristem but not in the founder cells (Jackson, D. Velt, B. and Hake, S. (1994) Development 120: 404; Scanlon, M., Schneeberger, R. G. and Freeling, M. (1996) Development 122:1683; Smith, L. G., Greene, B., Velt, B. and Hake, S. (1992) Development 116:21; Schneeberger, R. G., Becraft, P. W. Hake, S. and Freeling, M. (1995) Genes Dev. 9:2292). Ectopic expression of the maize knotted1 (kn1) gene (and related dicot genes) often leads to the organization of new meristems in dicot leaves but usually not in monocot leaves (Haraven, D., Gutfinger, T., Parnis, A., Eshed, Y. and Lifschitz, E. (1996) Cell 84:735; Sinha, N. R., Williams, R. E. and Hake, S. (1993) Genes Dev. 7:787; Lincoln, C., Long, J., Yamaguchi, J., Serikawa, K. and Hake, S. (1994) Plant Cell 6:1859; Muller, K. J. (1995) Nature 374:727; Williams-Carrier, R. E., Lie, Y. S., Hake, S. and Lemaux, P. G. (1997) Development 124:3737). Loss-of-function mutations in the maize kn1 gene result in defects in shoot meristem maintenance (Kerstetter, R. A., Laudencia-Chingcuanco, D., Smith, L. G. and Hake, S. (1997) Development, 124:3045).

In maize, several other genes belonging to the KNOX class have been identified. Two such genes, roughsheath1 (rs1) and liguleless3 (1g3) have been cloned and extensively characterized. Phenotypic analysis has suggested that both genes are involved in lateral organ development and are specifically implicated in retarding the acquisition of terminal regional identity. In particular, the dominant mutation of rs1, Rs1, results in unregulated cell division and expansion of the maize leaf (Schneeberger, R. G., Becraft, P. W. Hake, S. and Freeling, M. (1995) Genes Dev. 9:2292), while dominant mutation of lg3 results in the transformation of the blade into sheath and in the ectopic development of the ligule and auricle in the blade (Fowler, J. E. and Freeling, M. (1996) Dev. Genet. 18:198). Like other homeobox genes, the products of these genes are thought to be DNA binding proteins and are presumably involved in transcriptional regulation.

In light of the importance of homeobox genes in controlling plant development, it would be useful to identify the target sequences bound by KNOX genes, to manipulate gene expression in plants. The present invention addresses these and other needs.

### SUMMARY OF THE INVENTION

This invention provides recombinant expression cassettes comprising a homeobox binding site having a sequence at least substantially identical to ATTATTACATGNG (SEQ ID NO: 1) or ATTATTATTACATGNG (SEQ ID NO: 2) operably linked to a heterologous plant target polynucleotide sequence. The recombinant expression cassette may be incorporated in a recombinant plasmid or integrated into the genome of a transgenic plant. The heterologous target sequence can be used to encode a desired polypeptide or can be used to transcribe an inhibitory mRNA (e.g. antisense molecules, ribozymes, double stranded RNAs, and the like).

The invention also provides plants comprising the recombinant expression cassettes of the invention. The plant will typically comprise a gene in the KNOX class of homeobox genes, such as liguleless3 or roughsheath1. The homeobox gene can be an endogenous gene or can be introduced into the plant using well known techniques such as genetic engineering techniques (e.g. particle-mediated transformation, Agrobacterium-mediated transformation and the like) or by a sexual cross. The plant used in the invention is not a critical aspect of the invention. For example, the plant can be either a dicot or monocot. In the case of monocots, the plant can be a member of the family Poaceae, for example a member of the genus Oryza, Zea or Hordeum.

The invention also provides methods of controlling the phenotype of a plant. The methods comprise introducing into the plant a recombinant expression cassette of the invention. The expression cassette can be introduced into the plant using a variety of means including particle-mediated transformation, Agrobacterium-mediated transformation, or through a sexual cross. If the plant does not contain an endogenous homeobox gene, the method may further comprise introducing into the plant a homeobox gene that encodes a protein that binds the homeobox binding site. Exemplary homeobox genes are those in the KNOX class of homeobox genes, such as liguleless3 or roughsheath1.

The invention also provides methods of identifying a homeobox target gene sequence. The method comprise (a) providing a sample nucleotide sequence; and (b) detecting, in the sample nucleotide sequence, the presence of a homeobox target binding site having a sequence at least substantially identical to ATTATTACATGNG (SEQ ID NO: 1) or ATTATTATTACATGNG (SEQ ID NO: 2). The methods are conveniently carried using a computer. The methods may further comprise a step of making a nucleic acid molecule comprising the homeobox target nucleotide gene sequence and testing the ability of a homeobox gene to control expression of the homeobox target gene sequence.

Definitions

A “homeobox binding site” of the invention is a nucleic acid sequence that is specifically recognized by the homeodomain of a homeobox gene product, typically a member of the KNOX class of homeobox genes. The homeobox binding sites comprise the sequences ATTATTACATGNG (SEQ ID NO: 1) or ATTATTATTACATGNG (SEQ ID NO: 2) or sequences substantially identical (determined as explained below) to these sequences.

The phrase “nucleic acid sequence” refers to a single or double-stranded polymer of deoxyribonucleotide or ribonucleotide bases read from the 5′ to the 3′ end. It includes chromosomal DNA, self-replicating plasmids, infectious polymers of DNA or RNA and DNA or RNA that performs a primarily structural role.

The term “promoter” refers to regions or sequences located upstream and/or downstream from the start of transcription and which are involved in recognition and binding of RNA polymerase and other proteins to initiate transcription. A “plant promoter” is a promoter capable of initiating transcription in plant cells.

The term “intron” refers to a component of a gene's DNA and primary transcript that is removed during the process of mRNA biosynthesis. Regulatory sites are known to reside in introns as well as promoters.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

A polynucleotide sequence is “heterologous to” an organism or a second polynucleotide sequence if it originates from a foreign species, or, if from the same species, is modified from its original form. For example, a homeobox binding site of the invention operably linked to a heterologous coding sequence refers to a coding sequence from a gene different from that from which the homeobox binding site was derived, or, if from the same gene, a coding sequence which is modified (e.g. a cDNA sequence) from its naturally occurring form.

As used herein “operably linked” refers to the physical linkage between a homeobox binding site of the invention and a second sequence that allows the expression of the second sequence to be controlled by a homeobox gene whose gene product recognizes the binding site. The precise orientation of the binding site to the second sequence is not critical. For example, the binding site can be either upstream or downstream of the second sequence.

“Recombinant” refers to a human manipulated polynucleotide or a copy or complement of a human manipulated polynucleotide. For instance, a recombinant expression cassette comprising a promoter operably linked to a second polynucleotide may include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)) of an isolated nucleic acid comprising the expression cassette. In another example, a recombinant expression cassette may comprise polynucleotides combined in such a way that the polynucleotides are extremely unlikely to be found in nature. For instance, human manipulated restriction sites or plasmid vector sequences may flank or separate the promoter from the second polynucleotide. One of skill will recognize that polynucleotides can be manipulated in many ways and are not limited to the examples above.

A polynucleotide “exogenous to” an individual plant is a polynucleotide which is introduced into the plant by any means other than by a sexual cross. Examples of means by which this can be accomplished are described below, and include Agrobacterium-mediated transformation, biolistic methods, electroporation, and the like. Such a plant containing the exogenous nucleic acid is referred to here as a T₁ (e.g. in Arabidopsis by vacuum infiltration) or Ro (for plants regenerated from transformed cells in vitro) generation transgenic plant. Transgenic plants that arise from sexual cross or by selfing are descendants of such a plant.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The phrase “substantially identical,” in the context of two nucleic acids or polypeptides, refers to sequences or subsequences that have at least 60%, preferably 80%, most preferably 90-95% nucleotide or amino acid residue identity when aligned for maximum correspondence over a comparison window as measured using one of the following sequence comparison algorithms or by manual alignment and visual inspection. This definition also refers to the complement of a test sequence, which has substantial sequence or subsequence complementarity when the test sequence has substantial identity to a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. Because of the relatively short length of the homeobox binding sites of the invention, visual inspection can typically be used.

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al., J. Mol Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=-4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

(see, e.g., Creighton, Proteins (1984)).

An indication that two nucleic acid sequences or polypeptides are substantially identical is that the polypeptide encoded by the first nucleic acid is immunologically cross reactive with the antibodies raised against the polypeptide encoded by the second nucleic acid. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, highly stringent conditions are selected to be about 5-10° C. lower than the thermal melting point (Tₘ) for the specific sequence at a defined ionic strength pH. Low stringency conditions are generally selected to be about 15-30° C. below the Tₘ. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60C for long probes (e.g., greater than 50 nucleotides). Stringent conditions may also be achieved with the addition of destabilizing agents such as formamide. For selective or specific hybridization, a positive signal is at least two times background, preferably 10 time background hybridization.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions.

In the present invention, genomic DNA or cDNA comprising homeobox binding sites of the invention can be identified in standard Southern blots under stringent conditions using the nucleic acid sequences disclosed here. For the purposes of this disclosure, suitable stringent conditions for such hybridizations are those which include a hybridization in a buffer of 40% formamide, 1 M NaCl, 1% SDS at 37° C., and at least one wash in 0.2× SSC at a temperature of at least about 50° C., usually about 55° C. to about 60° C., for 20 minutes, or equivalent conditions. A positive hybridization is at least twice background. Those of ordinary skill will readily recognize that alternative hybridization and wash conditions can be utilized to provide conditions of similar stringency.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cased, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1× SSC at 45° C. A positive hybridization is at least twice background. Those of ordinary skill will readily recognize that alternative hybridization and wash conditions can be utilized to provide conditions of similar stringency.

A further indication that two polynucleotides are substantially identical is if the reference sequence, amplified by a pair of oligonucleotide primers, can then be used as a probe under stringent hybridization conditions to isolate the test sequence from a cDNA or genomic library, or to identify the test sequence in, e.g., an RNA gel or DNA gel blot hybridization analysis.

### BRIEF DESCRIPTION OF THE DRAWINGS

FIG. 1 is a diagram of the modified yeast one hybrid assays used to identify the sequences of the invention.

### DETAILED DESCRIPTION

This invention provides the first identification of homeobox binding sites in plants. The binding site sequences can be incorporated into any desired nucleic acid sequence. The expression of the gene will thereby be controlled by members of the KNOX class of homeobox genes, such as liguleless3 or roughsheath1. In addition, the binding site sequences provided here can be used to identify new or previously identified genes whose expression is controlled by homeobox genes.

Isolation of Homeobox Binding Sites

Generally, the nomenclature and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al., Molecular Cloning—A Laboratory Manual, Cold Spring Harbor Laboratory, Cold Spring Harbor, New York, (1989) or Current Protocols in Molecular Biology Volumes 1-3, John Wiley & Sons, Inc. (1994-1998).

The isolation of nucleic acids comprising homeobox binding sites of the invention may be accomplished by a number of techniques. For instance, oligonucleotide probes based on the sequences disclosed here can be used to identify the desired sequence in a genomic DNA library derived from any desired plant. To construct genomic libraries, large segments of genomic DNA are generated by random fragmentation, e.g. using restriction endonucleases, and are ligated with vector DNA to form molecules that can be packaged into the appropriate vector. The genomic library can then be screened using a probe based upon the sequence disclosed here. Probes may be used to hybridize with genomic DNA sequences to isolate homologous sequences in the same or different plant species.

Alternatively, the nucleic acids of interest can be amplified from nucleic acid samples using amplification techniques. For instance, polymerase chain reaction (PCR) technology can be used to amplify the sequences of related sequences directly from genomic DNA. For a general overview of PCR see PCR Protocols: A Guide to Methods and Applications. (Innis, M, Gelfand, D., Sninsky, J. and White, T., eds.), Academic Press, San Diego (1990). Appropriate primers and probes for identifying desired sequences from plant tissues are generated from comparisons of the sequences provided here.

Polynucleotides may also be synthesized by well-known techniques as described in the technical literature. See, e.g., Carruthers et al., Cold Spring Harbor Symp. Quant. Biol. 47:411-418 (1982), and Adams et al., J. Am. Chem. Soc. 105:661 (1983). Double stranded DNA fragments may then be obtained either by synthesizing the complementary strand and annealing the strands together under appropriate conditions, or by adding the complementary strand using DNA polymerase with an appropriate primer sequence.

Preparation of Recombinant Vectors

To use the homeobox binding sites to control plant gene expression, recombinant DNA vectors suitable for transformation of plant cells are prepared. The methods described below can also be used to prepare recombinant expression cassettes comprising homeobox genes, so that these genes can be introduced into a plant that does not naturally contain these genes. Techniques for transforming a wide variety of higher plant species are well known and described in the technical and scientific literature. See, for example, Weising et al. Ann. Rev. Genet. 22:421-477 (1988). A DNA sequence coding for a desired transcript or polypeptide, for example a cDNA sequence encoding a full length protein, will preferably be combined with the homeobox binding site and other transcriptional and translational initiation regulatory sequences necessary to direct the transcription from the sequence in the intended tissues of the transformed plant.

For example, a plant promoter fragment which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill. Such genes include for example, ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al J. Mol. Biol 208:551-565 (1989)), and Gpc2 from maize (GenBank No. U45855, Manjunath et al., Plant Mol. Biol. 33:97-112 (1997)).

Alternatively, the plant promoter, or a fragment thereof, may direct expression of the nucleic acid in a specific tissue, organ or cell type (i.e. tissue-specific promoters) or may be otherwise under more precise environmental (i.e. inducible promoters). Examples of environmental conditions that may effect transcription by inducible promoters include anaerobic conditions, elevated temperature, the presence of light, or sprayed with chemicals/hormones. Tissue-specific promoters may promote transcription within a certain time frame or developmental stage within a tissue. Other tissue specific promoters may be active throughout the life cycle of a particular tissue. One of skill will recognize that a tissue-specific promoter may drive expression of operably linked sequences in tissues other than the target tissue. Thus, as used herein a tissue-specific promoter is one that drives expression preferentially in the target tissue or cell type, but may also lead to some expression in other tissues as well.

A number of tissue-specific promoters, or elements that provide specificity derived from them, can also be used in the invention. For expression of a polynucleotide in the aerial vegetative organs of a plant, photosynthetic organ-specific promoters, such as the RBCS promoter (Khoudi, et al., Gene 197:343, 1997), can be used. Root-specific expression of polynucleotides can be achieved under the control of the root-specific ANR1 promoter (Zhang & Forde, Science, 279:407, 1998). Any strong, constitutive promoters, such as the rice actin or CaMV 35S promoter, can be used for the expression of the target polynucleotides throughout the plant.

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from desired genes will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta. The encoding polynucleotides that are used in the constructs of the invention are not a critical part of the invention. Any sequence whose expression is to be controlled by the homeobox genes can be used. Exemplary sequences include genes that naturally contain the homeobox binding sites disclosed here. For example, the maize Express Sequence Tag (EST) database was searched for genes that may contain the homeobox binding site. Approximately, 42,000 ESTs were examined and none were found to contain the binding site. This is not surprising since the binding site is predicted to occur in regulatory regions such as promoters, introns, and UTRs and the EST database primarily consists of coding regions. A similar search of the GenBank database did reveal several genes that contained a putative homeobox binding sequence. These genes represent putative targets of homeobox proteins. These genes include but are not limited to: Zea mays: liguless3, liguless4a, liguless4b, and alanine amino transferase and Oriza sativa: knotted2 (Oskn2). Examples of genes that naturally contain the homeobox binding sites of the invention include tissue and cell-type regulatory genes and genes involved in synthesizing plant growth hormones such as gibberellic acid and auxin.

In some embodiments the polynucleotide will be a heterologous sequence from a gene that is not normally under the control of a homeobox gene. Such sequences can be used to express desired mRNAs that inhibit expression of endogenous genes. Means for inhibiting endogenous genes using such techniques are well known to the those of skill in the art. Examples include antisense RNAs (see, e.g. Sheehy et al., Proc. Nat. Acad. Sci. USA, 85:8805-8809 (1988), and Hiatt et al., U.S. Pat. No. 4,801,340), ribozymes (see, e.g. Sun et al., Mol. Biotechnology 7:241-251 (1997); and Haseloff et al., Nature, 334:585-591 (1988)), sense suppression (see, e.g. Napoli et al., The Plant Cell 2:279-289 (1990); and U.S. Pat. Nos. 5,034,323, 5,231,020, and 5,283,184)) and double stranded RNAs (see, e.g. Waterhouse et al. Proc. Natl. Acad. Sci. U.S.A. 95:13959 (1998 and Fire et al. WO 99/32619). Alternatively, the heterologous sequence can encode a desired polypeptide. The function of the target endogenous gene to be inhibited or the expressed polypeptide is not critical to the invention. The sequences used in the invention can used to confer any desired trait on the transgenic plant comprising the constructs of the invention. Examples include genes that control development of organs (e.g. leaves) in the plant to control size, shape or other developmental features. Alternatively, genes conferring resistance to pathogens (e.g. bacteria, fungi, viruses and the like), environmental stresses (e.g. salt, drought, high temperature, anoxia, and the like) or pests (e.g. insects, nematodes, and the like) can be used.

Production of Transgenic Plants

DNA constructs of the invention may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment.

Microinjection techniques are known in the art and well described in the scientific and patent literature. The introduction of DNA constructs using polyethylene glycol precipitation is described in Paszkowski et al. Embo. J. 3:2717-2722 (1984). Electroporation techniques are described in Fromm et al. Proc. Natl. Acad. Sci. USA 82:5824 (1985). Particle mediated transformation techniques are described in Klein et al. Nature 327:70-73 (1987).

Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature. See, for example Horsch et al. Science 233:496-498 (1984), and Fraley et al. Proc. Natl. Acad. Sci. USA 80:4803 (1983) and Gene Transfer to Plants, Potrykus, ed. (Springer-Verlag, Berlin 1995).

Transformed plant cells which are derived by any of the above transformation techniques can be cultured to regenerate a whole plant which possesses the transformed genotype and thus the desired phenotype such as increased seed mass. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

The nucleic acid constructs of the invention can be used to confer desired traits on essentially any plant. Thus, the invention has use over a broad range of plants, including species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannesetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea. The methods are particularly useful for controlling expression of genes in members of the grass family (Poaceae), such as, Avena, Hordeum, Oryza, Secale, Triticum, and Zea.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

Identification of Genes Comprising the Homeobox Binding Sites of the Invention

Using the sequence information provided here, one of skill can identify genes whose expression is controlled by homeobox genes. This can carried by comparing the sequence of a gene to the sequences provided here and determining whether a homeobox binding site occurs in the sequence. This is typically carried out using standard sequence alignment and comparison algorithms described above. Thus, the sequences are conveniently carried out using a computer to compare sequence information. In the typical example, a database comprising a number of nucleic acid sequences can be screened for the presence of the sequences claimed here. Suitable databases are well known to those of skill in the art. Once a desired gene sequence is identified in a database, standard procedures can be used to prepare nucleic acid molecules comprising the sequence. The sequence can then be tested to confirm that the expression of the gene sequence is controlled by a homeobox gene.

### EXAMPLES

The following examples are offered to illustrate, but no to limit the claimed invention.

### Example 1

This examples describes the use of a modified one hybrid yeast screen to identify downstream targets of homeobox genes.

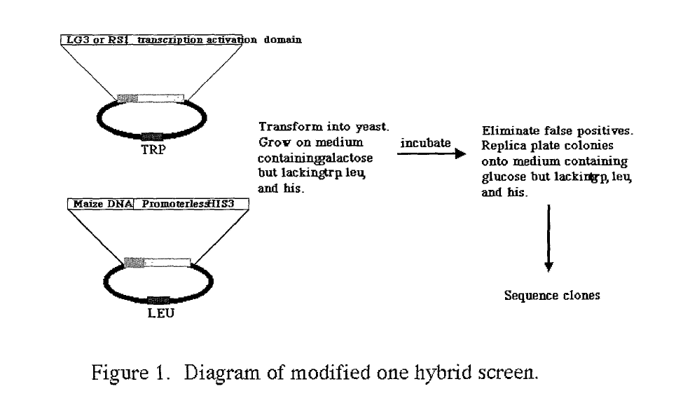

A modified one hybrid yeast screen (Li and Hershkowitz, Science 262:1870-4 (1996)) was used to identify potential downstream targets of the maize homeobox genes liguleless3 and roughsheath1 (FIG. 1). “Bait” plasmids encoding a fusion protein for either liguleless3- or the homeobox domain of roughsheathl were constructed using plasmid pJG4-5 (Gyuris et al., Cell 75: 791-803 (1993)) using standard molecular techniques and transformed into yeast strain YPH500 (MATα, ura3-52, lys2-801, ade2-100, trp1-Δ200, leu2-Δ1). These plasmids fused either the LG3 protein or the RS1 homeobox to a transcriptional activator. The expression of the fusion proteins was controlled by the carbon source in the growth medium such that galactose induced the production of the fusion proteins while glucose repressed production. These plasmids were maintained in yeast under Trp selection. Additionally, a library of potential targets was constructed by cloning gaspe flinte DNA, digested with Sau3A, upstream of a promoterless yeast HIS3 gene in plasmid pRS316-His (Wang and Reed, Nature 364:121-124 (1993)). These plasmids were maintained in yeast through Leu selection. Approximately 450,000 transformants expressing the LG3 fusion and containing a library plasmid were screened as well as 400,000 transformants expressing the RS1 fusion protein and a library plasmid. Transformants were plated onto a growth medium containing galactose and lacking histidine, leucine and tryptophan. Transformants were only able to grow into a colony if the HIS3 reporter gene was activated and if the cell contained both a bait plasmid and a library plasmid. Activation could have occurred by binding of either the LG3 or RS1 fusion protein to a target sequence from the library or by an endogenous yeast transcription factor. To eliminate the latter possibility, colonies were replica plated onto a medium containing glucose and lacking histidine, leucine, and tryptophan. In this medium, transformants do not produce the fusion proteins and hence formation of a colony is indicative of activation by a yeast transcription factor. Therefore, colonies that grew in the presence of galactose but did not grow in glucose were chosen for sequencing. Twenty four potential LG3 targets and 14 potential RS1 targets were partially sequenced.

Several orthologous and paralogous homeobox genes contained a highly conserved sequence in the second intron. These genes consist of lg3, lg4a and lg4b from maize as well as oskn2 from rice. Such high conservation across species and paralogs suggests that this sequence may play a conserved biological function. Because the sequence was identified in a an intron it was conceivable that this sequence may play a regulatory roll. The sequence data obtained from the one hybrid screen was screened to determine if this motif was present in any of the clones isolated. Four clones isolated from the LG3 (L27, L3, L26, and L36) screen and one clone from the RS1 screen (R39) contained the motif or a sequence very similar to the motif. Sequence from the clones identified in the screen are shown in the sequence listing. Three of the clones, L3, L26, and L36, were identical to each other over their full lengths. The area of conservation discovered not only included the motif present in the lg3 intron but additional flanking sequence.

### Example 2

This example shows how the homeobox binding motif can be used to find new potential targets for homeobox gene products.

The GenBank database was searched for genes that contained the homeobox binding motif using the algorithm PatScan. The Zea mays gene alanine amino transferase (aatl; GenBank accession number: AF055898) was found to contain the motif in intron 9 (SEQ ID NO: 6). The start of translation is marked with ATG and the homeobox binding site is underlined. Note that the aat1 motif matches the liguless3 motif for all 12 residues except that there is an additional nucleotide at position 12 in aat1.

### Example 3

A rice database was searched for genes that contain the homeobox binding motif. One gene, osk2, was identified. The OSK2 transcript in rice has been localized to leaf sclerenchyma and silica cells. The gene product is one of the two classes of protein kinases, that are presumed receptors that act at the cell membrane Takano et al. Mol Gen Genet. 260:388-94. (1998).

A number of maize EST clone #'s which are orthologs of the rice osk2 were identified. These are GenBank Accession Numbers AI740056, AI795751, AI861586, AI901790, AI943658, AI944187, AW017717,AW017996, AW018005, AW018231, AW065825, AW076310, AW076321, AW091047,AW120212, AW154941, AW163845, AW453387, BE344963, BE475880.

RT-PCR primers were constructed to specifically monitor the transcript from the maize osk2 gene. Comparing the expression levels in LG3− (normal) LG3+ sibling leaves showed a distinct reduction of the OSK2 transcript in LG3+ leaves as compared to the control transcript encoding alanine aminotransferase (aat).

In general, LG3 leaves under-produce sclerenchyma. Histological sections show that there are far fewer sclerenchyma cells, and each cell is less differentiated in response to ectopic expression of LG3. This result indicates that LG3 represses OSK2 which, in turn, causes less sclerenchyma proliferation and differentiation.

Twelve other target genes were identified in maize and further examined by RT-PCR. The ZmDB (see, e.g., www.zmdb.iastate.edu) accession numbers in of ESTs corresponding to these genes are set forth Table 1, along with the exact target sequence in the rice ortholog.

TABLE 1

| | | |
|---|---|---|
| 025530 | RNase L inhibitor | TTTATTACATGTC |
| 195254 | myo-inositol-1(or 4)- | TACATGTAATAAA |
| | monophosphatase | |
| 052623 | putative ABC transporter | TTTATTACATGCC |
| 003077 | Oryza sativa mRNA for OSK2 | CTTATTACATGTT |
| 048326 | putative ubiquitin-conjugating | TTTATTACATGAC |
| | enzyme | |
| 005709 | subtilisin-like serine protease | TTTATTACATGTT |
| 004768 | zinc finger protein | CTCATGTAATAAC |
| 011198 | CoA-thioester hydrolase | ATTATTACATGAC |
| 001355 | O-sialoglycoprotein | ATTATTACATGAA |
| | endopeptidase | |
| 010124 | glucose-6-phosphate/ | CACATGTAATAAA |
| | phosphate-translocator | |
| 006782 | P-glycoprotein-like | CTTATTACATGCT |
| 009018 | glutaredoxin | CTCATGTAATAAT |

Other maize genes that are targets of the LG3 gene include CDP-diacylglycerol synthetase, MOM(gene silencing), Pspzf zinc finger protein-like, NBS-LRR type resistance gene, calcium-dependent protein kinase, S-receptor kinase, SNF2/SWI2 family global transcription factor, putative DNA-binding protein, lipoamide dehydrogenase, SEC 14-like protein, auxin transport protein, GDSL-motif lipase/hydrolase-like, and wall-associated kinase 4.

It is understood that the examples and embodiments described herein are for illustrative purposes only and that various modifications or changes in light thereof will be suggested to persons skilled in the art and are to be included within the spirit and purview of this application and scope of the appended claims. All publications, accession numbers, patents, and patent applications cited herein are hereby incorporated by reference in their entirety for all purposes.

What is claimed is:

1. A recombinant expression cassette comprising a homeobox binding site having a sequence at least substantially identical to ATTATTACATGNG (SEQ ID NO: 1) or ATTATTATTACATGNG (SEQ ID NO: 2) operably linked to a heterologous plant target polynucleotide sequence.

2. The recombinant expression cassette of claim 1, which is incorporated in a recombinant plasmid.

3. The recombinant expression cassette of claim 1, wherein the heterologous target sequence encodes a polypeptide.

4. A plant comprising the recombinant expression cassette of claim 1 and a homeobox gene that encodes a protein that binds the homeobox binding site.

5. The plant of claim 4, wherein the homeobox gene is heterologous to the plant.

6. The plant of claim 4, wherein the homeobox gene is liguleless3.

7. The plant claim 4, wherein the heterologous target sequence encodes a polypeptide.

8. The plant of claim 4, wherein the plant is a member of the family Poaceae.

9. The plant of claim 8, which is a member of the genus Zea.

10. A method of controlling the phenotype of a plant, the method comprising introducing into the plant a recombinant expression cassette of claim 1.

11. The method of claim 10, wherein the recombinant expression cassette is introduced into the plant using particle-mediated transformation.

12. The method claim 10, wherein the recombinant expression cassette is introduced into the plant using Agrobacterium.

13. The method of claim 10, wherein the recombinant expression cassette is introduced into the plant by a sexual cross.

14. The method of claim 10, further comprising introducing into the plant a homeobox gene that encodes a protein that binds the homeobox binding site.

15. The method of claim 14, wherein the homeobox gene is liguleless3.

16. The method of claim 10, wherein the plant is maize.

17. A method of identifying a homeobox target gene sequence, the method comprising:
(a) providing a sample nucleotide sequence; and
(b) detecting, in the sample nucleotide sequence, the presence of a homeobox target binding site having a sequence at least substantially identical to ATTATTACATGNG or ATTATTATTACATGNG, thereby identifying a homeobox target gene sequence.

18. The method of claim 17, further comprising making a nucleic acid molecule comprising the homeobox target nucleotide gene sequence.

19. The method of claim 18, testing the ability of a homeobox gene to control expression of the homeobox target gene sequence.

20. The method of claim 19, wherein the homeobox gene is liguless3.

21. The method of claim 17, wherein the sample nucleotide sequence is provided in computer-readable form and the step of detecting is carried out using a computer.

22. The method of claim 17, wherein the sample nucleotide sequence is from a plant.

23. The method of claim 22, wherein the plant is maize.


SEQUENCE LISTING (18 sequences)

SEQ ID NO: 1 - DNA, 13 bp, Artificial Sequence
  Description of Artificial Sequencehomeobox target binding site
attattacatgng

SEQ ID NO: 2 - DNA, 16 bp, Artificial Sequence
  Description of Artificial Sequencehomeobox target binding site
attattattacatgng

SEQ ID NO: 3 - DNA, 580 bp, Artificial Sequence
  Description of Artificial SequenceL27 clone isolated from liguleless3 (LG3) screen
gatcaaacactcacttgtaattgaggcaagaggcaccaattgtgtggtggcccttgcgggaagtttgattcccaagtgatttgagaagagaagctcactcggtccgagggaccgattgagagagggaagggttgaaagagacccggcctttgtggcctcctcaacggggagtaggtttgcaagaaccgaacctcggtaaaacaaatccgcgtgtcacactcttcatttgcttgcgatttgttttgctccctctctcgcggactcatttatatttctaacgctaacccggcttgtagttgtgattaattttttgagaatttcagtttcgccctattcaccccccctttaggcgactttcagtctattcacttcatacctaagtaccatgtgagaaatcctagacaagtttacaataatgttgtcacacatgcaatctaacaatagcatgagcatcgcccaccttcacatcgagcaacaacagtcaagcaactacacatcaacattgtgaagatgtcgaaacttgaaacacctagatgctcaacatcattctggggactaanaccaagtgagaaagaaaaga

SEQ ID NO: 4 - DNA, 2041 bp, Artificial Sequence
  Description of Artificial SequenceL3, L26 and L36 clones isolated from liguleless3 (LG3) screen
gatcgtgggcgtgtacgggctgcagcagtcggcgctggagacggaggaagcgctgagccagggcctggaggcgctctaccagtcgctgtctgacaccgtggtctccgacgcgctcagctgcccctcgaacgtctccaactacatgggtcagatggccgtcgccatgaacaagctctccacgctcgagggcttcgtcagacaagtaagtaaccaaggccgttggccgatgcatccgatcctatcatcgaccggtataccttttgatctctagtaccgggaaaagtcagtaaagattcttgacccagcagtccgaaggacccgcaactcagaagtgagactctgtaaggttcagtggagcagagaaggagaagaggaagccacctgggaaagtgaggactctctgaggagagaataccgttaccttttctcaaaccccgtctgaatctcgagggcgagattctttwagtggggtagggtttgtaacatcccaatttctaacccaggtttgaaaccctaatctaaccccccttcttattatctattcccaaaacttccctttagaattctttcccaaacatatgcatacactttgtttgaatatgaacacctcacttagattttatttttctagcaccttagattattytattaaaattaattgttcaaaagaaaagaatataaaaaatggatataggaattagaaaataaaaagaaaaagagaaaacccaccctagctgggctgaccgcgaccgcctttcctccctcgcgcccgcgctttcccccctttcggcccattgtcggcctgctcctgcgcgcgcagccgctccgcgctccctctctctctctcgctgacgagccgaccccacctgccagccgctctctctcgcgcgctcactcccgctggcagaccggcccgaccagtcagccgcttcgtcgtcctcctcgcgtacggctcatcgcggtcacctccgaccgctgtcacctccatcgcccccatcccctcgccgcacaggagagtttggcaccgccccatcctcgccgcttgaccgcgtccttgcccggtaccgccaccaccgtgaggtctcgtcgtcgccgctgtgcggcattaatgccagcgctgtgcacctcaccggcgcccgccgagctccgctgctccccttccctcgggcgcctataaaaaggtcgccccaagcacctcctcccccgcaccggccttagccaccactctcctccctcacntgagcccatttcgcggaagcgccgccgtcgtctccctctccggtaagccttcccctcatctcccttcccctctgttggaccaacgagcaattacttaggcctaccagcttcgccacaccgctgcgaactcaggacaccactcccccactccagtcccttgcccgaagctcaccgacgacgactcttgccgcggagctctgccacctccccgcggacagccccctcccggccccctctagctaaattgagcccacctctaggttcaccagcccctccccgtgataaggcacatacccgttgtccaagaactgggccactggcggccgggttctccccgcggacggccggttcgccccgccgtcaaccgtagcgcccccccttccgctctcgctggcgcgtgggccccgcggcgacggcgtcatcccctcgcgcgcccacatcgtcacccgccttgggccgcagctgggccggcgccctcgcgcccccgcgtgcgctgcgcctggcttggcctggctgggccagaaatccccccggcccaccagtgctgaaattccttttctttttcctttttcagccatttctcctctaattagtatttctcaatattttatgcaccaaaaattatcaaaatgatttctaaattcacatgtaataataatgttagaaaatgacacactacaattagtaaaccactgttgatgtattgttttattgactgttttactagaagaagcggggaccgttgatccggaacccgtagccccggaagaagggccggagcccgatctcccggaagtagatc

SEQ ID NO: 5 - DNA, 4155 bp, Artificial Sequence
  Description of Artificial SequenceR39 clone isolated from roughsheath1 (RS1) screen
gatctctcatatgattcacactaatcgatttataatatagtttaatattcaaaccacatatatcttcagtaaattatagctccgtaaccgtaactccgattttagtggttctcgaactcacgatatcgtagcatcgcgtaaattattattacatggttcattcttatgtttggtgtgatgttaattttgcctataccatgtttgtttgtattgctacgactagcaatgaggtcacgtgtcatctgaagagcaagttggtacctggaatctcaagtcccaggcaagttgtgcccttgatcacatagacatgctaattttattgaacatcactcttttgtttcttgtgttgagcctacttgtatagatgaggcgctacaggatctctgggacacagggagcaaccagacagcggggaattcctcaccggaagcacttctccgccgcgggccgcactgctccagaggtggattatgtcaaagacgccattgaaggtaataagcctcgtgatctgtggaattagttttcctttctgaacttggktcctctctttcagacacttgtgtactgttgtcttcattctccaatggtctaaataagggggtgtttggtttatttagtcacccttctaaactttagaaactaaactttagtccctacaatttctatttgggaggtgcctttaatcatattgattggcagtttagggactaaaagtgtatccctcttttgaatgggctggatttgcaggctcccccgtgggctttcctcaatccgtcctcttttgctcactcttatcctttctccgttccaaattctatacatttgcgaacacatggttgatatgtgctcttatggtttagattatgatgagtgattgtcaacaagtgtaacgtcttgggttgagttgtgtggactaactagggcatggatgtgattatgtaacatgtcttttggcttatgatgtgcaggtgtggagtctggagacaatggtcaatgaccaagttcatatgggttcatgtcgaggggatctccgcagtccgaagtcttcatcggacccgatcgaagacttcgaagtgcctgcatccatcacgggacaaggcattcatggctcccccacaggtgatgagcgaagcgaaggacatgagacccgtcacgatcgatggaagcacctccagctcctcctgcagccacccgagaaagcagaggtccaccttcttaccatatgcaatagagggggccgtacgcgcaccaaactaccggtacgcgtccacaagcaacgcatgtgctcagtccacccctgtgcgggtagaggcctccgccttatccagatagccgtgaaggctcctgtggttcgcctctgcctccgcagcacgttggagggcaaggctagcctcggcgtgtgcaagtttggcatcgacgcgcgcagcctgggcttcgcgcaggccttgtcagcatcctccttctctgctaccagccagcggccgagttcctccttttcggccttcgcagccgacagcttatccattaggttgcggctcttgttcttcgcggccgacttgtcccgcacaacctcaatgtgctgcgtccggaagcgctcaatcctcccctcgagtcatcgcctttcggcagcgaactccaaggtcagggcccccggacgcaatccgctcagcgaaggcggccgcctgtcatcaaacaccatcaaattcagcgtcacataagaagaacatactgtcaaagactacttacgtgtttcagctgctgcgtaaccgagcccaccacgtccttggcagtggcggccgatgcgatctcgccgctggcgcaaaacttggccaaaggatcaagcacgagttctccgctccctacacaccacagcaaaatggtgtggtagagaggaagaacaggacgcttatagacatggcgaggacgatgcttggagagttcaagacccccgagtgcttttggtcggaagccgtgaacacggcttgccacgccatcaacagggtctaccttcatcgcctcctcaagaagacgtcgtatgagctactaaccggtaacaaacccaatgtttcgtattttcgagtttttgggagtaaatgctacattctagtgaagaagagtaggaattccaaatttgctccaaaagccgtagaagggtttttgttaggttatgactcaaatacaaaggcgtatagagtcttcaacaaatcatcgggtttggttgaagtctctagcgacgttgtatttgatgagactaatggctctccaagagagcaagttgttgattttgatgatgtagatgaagaagaagttccaacgaccgcaatacgcaccatggcgattggagatgtgcggccacaggaataagatgaacgagatccgaatccgaaactctttccttccttgcacgtcctaactttaacagtcataccggaaggagtcaggccaccgccatgtccaaaccggacaaatctttccccctccttatcctgccggtgcttccccarccttcataaccctggggttgggtcgtacgagttcagatcgagtggctgcccacacagcctcgagtggttgttctttttatgagtataggtaatgaaagatgacaagtcggtccttatacgagaggacaatccttctgctcacgcctaaaccagctgagccatcaccttaggccctctcctaaaccagggagtccctgatcatcctactcaccaagtgatgagagtgaatacccttcatcgcacacttttttggaaacatgttacttgcacccttttacttatcccatgtcttgtaatcaaaggtattagttaatgcgggctctctcatgctcaccatgcaattggattccatcccatattccaggcttaggtagtggtagagagaaataggtaaataatgcatcaagggaaggatggacttgccttcgtcgaagctttcctggcacaagaggtttatctcagaggggtcgggttcttgcccttctttcggagctataaattccggaggatcggacccctcttccgctawtcaaacmcagataatamcnaatacatcaatcatggtgactttagtggggtgtgccatttcttatatctcattattttggtgccctataatttatttgaactatttttggtacataaaatattagcataaagttctatatgagaaaaatgggaaaataaaagaggaaataaaaagaaaaagggtttcctgccttgctgggccgggggggggggcagatttcggcccacccgggcgcgagcgcgcgcgcgcgggcgcggctggcggcccagttggcccagcagcgagggacgggtgggggaaccgacsctgcggtgcgggggcccacatgccascgagggggagggtgcttaacggcgcgggcggtaacggagggagggggagttcgaccggggtgggaattcgcggcggttctccgccgtgggtccggttccgcagcggggaggtggtggtgctgcacgggcgggggcaggcgatcatccccaggaagcgcgtccaaccccttggacaactcgatcttcccgtctgcttcggaacaccnatncaacttccgaagggagaccctkaagttcraggtggtcgggttccgaggaacctaccacgcgggtattgggraggccctactacgtgaagttcatggccgtccccaactacacctacctcaagctcaagatgctgggccccaacggggtcatcaccgtcggccccacgtacaaacacgcgttcgaatgcgacgtggagtgcgtggagtacgccgaggccctcaccgagtctgaggccctcatcgccgacctggagagcctctccaaggaggtgccagacatgaagcgtcatgccggcaacttcaagccagcggagacggttaagtccgtccccctcgaccccagcagcgacgcctccaagcaagtccggatcggctccgggctcgatcccaaataggaagcagtgctcgtcgactttctccgcgcgaacgccgacgtcttcgtgtggagtccctcggacatgcctggcataccgagggatgtcgccgagcactcgctggacatccgagccggagcccaacccgtcaagcagcctctgcgtcgattcgacgagaagaagcgcagagccataggcgaggagatc

SEQ ID NO: 6 - DNA, 6176 bp, Zea mays
  alanine amino transferase (aat1)
gattgaccggggcatggaggtagcagactcaggtaaaaagtatcatggttggccagaacaggacccggttttaatttttcccatatttttttaataaaagtctatattatacttgagtataaaatataaaacgaaaaatatgtgtcatgttggctagatagatgtgagtttaaagcaaacaaccatagttcaaatgacattcacatttacatacttgcacggctgcgcacatgcaagctttgcatggagcggagacgtttgcatgcatggttggatgcaccagtttagttgcatacttgcatgcgtgatgtctgtgctgatttaggggttacatgtagactggaaccggcttcatgggtggccacgcatggggacgccacgcagaggattgcatgcacgcagaggacggagcgggggcacgactgtggtctacattagactcttaatagttagtagagatttaaattatcaaataactacatatagattataatatgtctagattataatctagattatataatttataagctgaaacaaatatgtcctttgtaatgttttacttagcaaatttgtgagagagaaaagaagaaaaagcaagaaaacatccgacggcgtcacatcacaagaaatagaaaaacaactcacatagatgaatcggcaccgaaacgaaagctaagtatatatataaaaaagtgccaaaattgcaatcctccatcttctaaaataaagaatatagcacgaggaaattggcaagcaagtccatcttcagcttccatctgagacattttttattgcaaaaaccagagagtgattttttttttttgttttcaaaaccgcggcagtggtcggcggccccccggtctccttccactcgcttttccggcgggcgactggcctaaaatcgcctcttgtttgtttgcttccgtcgtcccctctccacgtagagcacgcccccggaccaccttttataagcaccaaccatctggccttgccgcgccgattcgccacgaagccgcactgtgagagagggacgcggcctgttgtcccggcttttagacgcaacttccatcgctgcgaagctatccattcgtgttcgtcatggccgccagcgtcaccgtggaaaacctcaaccccaaggtgagatcgccgcccgttttctcccccttgtctagctttctggtccttgaaggtcccatttttgatggatttttcggtgggaagtttgggttttaacgagatggaggaggctctgtggatcttctgcgtccctctgcgtgtccctgattcagcttagccgcgggttggactctcgtgtgattgagaaatttggggtgaaaagattatccttgatttggttgagcggtacaagtcgtgcttagttcttgttcttgatgatctgattttgatttatacttataaatcaaacgataaaatttttaggataattgtaacgactgtaccggtgcaaagttacatcgtttagatgcttgcaataagtgcagttccttatacccagatcactagtcgctaactcgctatcgctctctctcaaccaagagttagatctcagcgcgactgcgattcgagctgctatagtgccttttctttttctttttcttttgttttgtcgtgagtcgtgaccgaatagtttaagtcatctgtcagtatcctttcccccctttgactcaaaaggggatatggcgatttgtttggactgctgccccacttctgttgcacaatatatataggttgatgctgtattgtttctgtgagatctgactatctgaggtactactctgttggcaatctgtgctttgttactaaacctattttgtaaacctgctgaatgtcgtgtgatcatgatttccatctgtttcaaagacaacagctttcgtgctttgcttctctgggtaaggcgagtgtggcttctcatgcctagctaatacactaataactattacaggtcttaaaatgtgagtatgcagtccgtggggagattgtcatccatgctcaggtatcttgactactgatgttcaacaaggcttgcagtctagtacttcactatgttatatatcactgtgttcatgttccccaaagaatcgtttattcaccttatcacaaggcaatacagttaaatcaggtaatgttcatatagagttttgttagactgttgtagtaatagtatttgcttttgacactgctatctgacaaagaacagcatagtgcagtacctgaatttacaaacatgtatttgtgttatagagcttactttatagctttcgaagtctattttggtacaccaaagtaattaagcatgtagtctacttgattgctaataaccatattgctttattagtaatttaatttgcaattgttccctgatgaaattcttcctaattaattgaaggtcaataatctttttcaccatatattatatgtctcattgtgaatgcttttaattaagaatcttgtgcgctccatgccactggcagatctgccattaaggatctgcttcgaaattgatttaatgtagccccattgtttttttctgttaacagcgccgccagcaacaactacaaacacaaccagggtctcttccttttgatgaggtactaactgttgatttaaaagttcattgaaagtacctctactttcctttttttttcaaaaattatatagtccgttccaattatatttgtaccctcgcagatcctctattgcaatattgggaatccccaatctcttggtcagcaacctgtgacattcttcagggaggtatgtgtttattttacctgcagattccacctttgtttcttctatatttacatggtaccttgcaggttcttgctctttgtgatcatccgtgcctcttggaaaaagaggaaaccaaatcattgttcaggtgaatatcatcatcattcaatttacagaactcctaaattcagagatctagtgtgtgatatcctgctatttttcccaacttcagtgtcaaagcaacctaaaaattctaaaaaatacagagatagggcaatctatcttcctttaaaatcaaagctgtggcattttctttgaaattagtaaacatttatataaatagtaaaatttcctggagatctgtcaggtacttaactttttcccttcaacttccacagtaattaaacatacctaggaagatagttttggagttctcatgtttaattgatttgttcatcagaagaaccattacatccgtgcctaattatgcatgccctttcatttttcctaaaatttcccttgataccatttcaagttgcaaagatgatttttttttgcccgtactgtataatatttttgttagccataaactttcaaaagtagttccagtgtcccatttatacataaattcttatgtgtacttgatgggtcgtctcactgcagtgctgatgctatttctcgagcaaagcagattcttgcaactattcctggaagggcaacaggtgcatatagtcacagtcaggttggcatctcactctgatatatttattgaccacaatgagagaggagtcttggcttcatttgatttttttaatgtttcctagggtatcaaaggactgcgtgatgcaattgctgctggaatcatgtcacgtgatggattccctgcaaatgcagatgacattttcattacagacggagcaagtcctggggtgtgcatttttaacttctctctcaattgtcctggacattttctgaataaacatgatatcactgaagtctcatcttgaccctattcttccttcttaaggtccacatgatgatgcaattactgattaggaatgagaaagatggcattctgtgcccaattccccagtaccctttgtactcagcctccatagctcttcatggtgggactcttgtatgtactttcatttgctcatcagaaagaatatggtctatagtactaaatattgtatcaccgacaccgaaaatcgagcgcatgttctttcaggtcccctattaccttaatgaaaaaaatgggtggggtttggagatttctgattttaagacgcgactggaagatgttcggtcaaaaggcattgatgttagggccctggtggttatcaatccaggaaatccgactgggcaggtttgtactaatcatttctcctctctcgttaaagaacatatctgcttgcttagatattctatttcctatgcacaacatacttctttataattgagtgttattacatgaagtgtgtatttcttgtactcccgtttttaagttagtgaatactttgtatacttattttgtgcagggaaaactttttgcgtagaaaaaaatgtggtttttatcattctgacttctgaaatgtgaatgaatgctttttcaatgccggtgaccaaaacacaaattttattgaagtggttcatttatttgtaggttcttgctgaagacaaccaatatgatatagtgaaattctgcaaaaatgagggccttgttcttctagctgatgaggtgagtgataggcaagtagctgatgtttagacgtcagaacaaccacaaaaaatttgccacggcctctaacactaatgcccattttcattgttaggtatatcaagagaacatctatgtggacaacaagaaatttaactctttcaagaagattgtaagatccatggggtatggcgaggatgatctccctctagtatcgttacagtctgtttccaaaggtagatttatatagccagctacgttatctgccgtttggtcaagtaactggcttatccaaaagattttgctgctgcaaccaggatactatggagagtgtggtaaaagaggagggtacatggagattactggcttcagtgctccagtaagagagcagatctacaagatagcttcagtgaacttgtgctccaatatcactggtcaaatccttgcgagcctcgtcatgaatccaccaaaggtctgccatatggaattctttaaattccgccctggttggaattaggaaatgccaaatcgtccggttaaaaatcgttgctttattctccaggctggggacgaatcatatgcttcctacaaggcagagaaggatggaatccttgagtctttagctcgtcgtgcaaagttcgcctggttgcatttccactacattcttgattgtcttctttaaaaatattcaagatttttcttttgttcgttttacaggcgttggaggatgcattcaacaagcttgagggattttcatgtaacaaggccgaaggagcaatgtatcttttccctcagattcatctgccacagaaggcaatcgaggctgctaaagctgctaagaaagcgcctgacgctttctacgctctccgtctccttgaatcaactggaatcgtcgtggtccctggatctggatttggtcaagtgagtcaactcaaccttcacatactgtaatatacttgtgcgcctggcctgtttggttcgtggcgcataggatagtgggttcttggcgatgcatttttcatgtaaatcagtcagacagtcacagctacttcgtttcatgttaaccccgtcctaacgatcgacggtccgctcctttgctggtacttcgcaggttcctggaacatggcacatcagatgcacgatcctgccgcaggaagacaagatccccgcggtgatcagccgcttcagggctttccatgaggcgttcttggccgagtaccgcgactaaactgtttacgctgtgtgtgaagttaagttggaaggtttcagtagaactgtagaagagctcagtgacagcgcagaaagaggcagtgttagaaaggcttaggtccttcttcacaggagcaacagttgtcttgtctccatcaaggagaaataaaaattttagttttacaacttctcattgacactgttgggttcttgacatggtagagaattgtgtgcactgtgcctggcactgggtcctactagcggcgcaatttaagcgtgaccaggatggcgccatcagccatctctgtcgatattacactgctagctgaaaatgccacttggtcctttcaggtgtttgtggaccaaaacctaagttgccttccttcaggaacttgtggactgaaagcaatagatgcgacctgaatgacggttctttgtggatccgaattcttcgccctatagtgagtcgtattcacgcggccgcgtcgac

SEQ ID NO: 7 - DNA, 13 bp, Artificial Sequence
  Description of Artificial Sequencerice ortholog homeobox binding motif from maize RNase L inhibitor EST 023330
tttattacatgtc

SEQ ID NO: 8 - DNA, 13 bp, Artificial Sequence
  Description of Artificial Sequencerice ortholog homeobox binding motif from maize myo-inositol-1(or 4)-monophosphatase EST 195254
tacatgtaataaa

SEQ ID NO: 9 - DNA, 13 bp, Artificial Sequence
  Description of Artificial Sequencerice ortholog homeobox binding motif from maize putative ABC transporter EST 052623
tttattacatgcc

SEQ ID NO: 10 - DNA, 13 bp, Artificial Sequence
  Description of Artificial Sequencerice ortholog homeobox binding motif from maize OSK2 EST 003077
cttattacatgtt

SEQ ID NO: 11 - DNA, 13 bp, Artificial Sequence
  Description of Artificial Sequencerice ortholog homeobox binding motif from maize putative ubiquitin-conjugating enzyme EST 048326
tttattacatgac

SEQ ID NO: 12 - DNA, 13 bp, Artificial Sequence
  Description of Artificial Sequencerice ortholog homeobox binding motif from maize subtilisin-like serine protease EST 005709
tttattacatgtt

SEQ ID NO: 13 - DNA, 13 bp, Artificial Sequence
  Description of Artificial Sequencerice ortholog homeobox binding motif from maize zinc finger protein EST 004768
ctcatgtaataac

SEQ ID NO: 14 - DNA, 13 bp, Artificial Sequence
  Description of Artificial Sequencerice ortholog homeobox binding motif from maize CoA-thioester hydrolase EST 011198
attattacatgac

SEQ ID NO: 15 - DNA, 13 bp, Artificial Sequence
  Description of Artificial Sequencerice ortholog homeobox binding motif from maize O-sialoglycoprotein endopeptidase EST 001355
attattacatgaa

SEQ ID NO: 16 - DNA, 13 bp, Artificial Sequence
  Description of Artificial Sequencerice ortholog homeobox binding motif from maize glucose-6-phosphate/phosphate-translocator EST 010124
cacatgtaataaa

SEQ ID NO: 17 - DNA, 13 bp, Artificial Sequence
  Description of Artificial Sequencerice ortholog homeobox binding motif from maize P-glycoprotein-like EST 006782
cttattacatgct

SEQ ID NO: 18 - DNA, 13 bp, Artificial Sequence
  Description of Artificial Sequencerice ortholog homeobox binding motif from maize glutaredoxin EST 009018
ctcatgtaataat